SEQUENCE LISTING (4 sequences)

SEQ ID NO: 1 - DNA, 1739 bp, Corynebacterium glutamicum
  CDS (261)..(1520): pepC-Gen
caccaacgtgcctttgacctgggcgacggtcttaggctctggcaggcgatcaatcgcgcggaagcgatacaacaggggacactgctggtaatccccggcgcgcgacggtgacagcgccaatgggcgtggctttttcttaacgttttcaactgggctggtcataatgatcacctactttaacggcttcaggtgacattgtggattcgcattgtggattcgggggcccgcgctgtttccaagaatttggctacccttgttctatgcatgtaactgacgatttcttaagttttattgccctaagcccaagttcctatcacgcggccgcggcggtggagcgcaggttgctccatgaggggttcattcgtcaggaagataccgatgaatgggatgcccgccctggtgggcatgtgacggtgcgtgggggagcagtagtggcgtggtgggtgcctgaggatgcttcgccagattccgggttccgcatcattgggtcacatactgattcaccgggtttcaagttaaagccccgtggggatctttcctcacacggttggcagcaggccggcgtcgaggtttacggcggaccgatcctgccaagctggctggatcgcgagctggccttagcaggccgcattgtgcttgccgacgggtccgtcaagcttgtcaacaccggcccgattctgcgcatcccgcacgtggctattcatttggaccgtactgttaattcccaactcacccttaatccacagcgtcacctgcagcctgtgtttgctgttggtgagcccgacgtatcaattctggatgtcattgctggtgctgcggtagtggatcctgcagatattgtcagccatgatctgatcacggtggctacccaagatgctgaagtatttggcgcacatggggatttcttggcgtctggtcgcctggataacctgagcagcgtgcatccatccatgactgcattgattgcggcttcgcaatctgacgatactggttcggatattttggttcttgctgcattcgatcatgaagaagtgggaagtaattccacctcgggtgccgggggccccctgttggaggatgtgctcaaccgtactgctcgtgcgttgggtgcagatgaagatgagcgacgccggatgtttaaccgttccaccatggtctcagctgacgcggcacactccattcaccccaacttccccgagaagcatgatcaagctaattaccccatcattggtaaaggtcctgtattgaaggtcaacgccaaccagcgctacacctccgatgcagtcacttcaggcatgtggatcagggcatgtcagattgccggtgtgccacaccaggtgtttgccggcaacaacgatgtgccgtgtggttccaccatcggcccgatcagtgcgactcgcctgggtatcgattctgtcgatgtcggtattccattgctgtccatgcactccgcacgcgaaatggccggagtgaaggatctgatgtggtttgaacaagccctggaagcctatctggtaaattaacgccgagttcaatcaagacaagcacacagaagaaagtgagggctcatgccctactcaggtccgttccaagcaggcgaccgcgttcagctcaccgacgctaaacgccgccatttcaccatcattttggaaccaggaaccacctaccacacccaccgtggacaaatcgcacacgatgacatcatcggcgccgatgagggcactgttgtccactccacc

SEQ ID NO: 2 - protein, 420 aa, Corynebacterium glutamicum
MHVTDDFLSFIALSPSSYHAAAAVERRLLHEGFIRQEDTDEWDARPGGHVTVRGGAVVAWWVPEDASPDSGFRIIGSHTDSPGFKLKPRGDLSSHGWQQAGVEVYGGPILPSWLDRELALAGRIVLADGSVKLVNTGPILRIPHVAIHLDRTVNSQLTLNPQRHLQPVFAVGEPDVSILDVIAGAAVVDPADIVSHDLITVATQDAEVFGAHGDFLASGRLDNLSSVHPSMTALIAASQSDDTGSDILVLAAFDHEEVGSNSTSGAGGPLLEDVLNRTARALGADEDERRRMFNRSTMVSADAAHSIHPNFPEKHDQANYPIIGKGPVLKVNANQRYTSDAVTSGMWIRACQIAGVPHQVFAGNNDVPCGSTIGPISATRLGIDSVDVGIPLLSMHSAREMAGVKDLMWFEQALEAYLVN

SEQ ID NO: 3 - DNA, 18 bp, Corynebacterium glutamicum
  Primer pepC-int1
ctttcctcacacggttgg

SEQ ID NO: 4 - DNA, 19 bp, Corynebacterium glutamicum
  Primer pepC-int2
tcccacttcttcatgatcg

(19) United States
(12) Patent Application Publication
(10) Pub. No.: US 2002/0098554 A1
(43) Pub. Date: Jul. 25, 2002

(54) Nucleotide sequences coding for the pepC gene

(76) Inventors: Mike Farwick, Bielefeld (DE); Klaus Huthmacher, Gelnhausen (DE); Brigitte Bathe, Salzkotten (DE); Mechthild Rieping, Bielefeld (DE); Walter Pfefferle, Halle (DE)

Correspondence Address: PILLSBURY WINTHROP LLP, 1600 TYSONS BOULEVARD, MCLEAN, VA, 22102, US

(21) Appl. No.: 09/804,073

(22) Filed: Mar. 13, 2001

(30) Foreign Application Priority Data
Sep. 19, 2000 (DE) 100 46 229.4
Feb. 23, 2001 (DE) 101 08 828.0

(51) Int. Cl. C12P 13/04; C12P 13/08; C12N 9/00; C07H 21/04; C12N 15/74; C12P 21/02; C12N 1/21

(52) U.S. Cl. 435/106; 435/69.1; 435/183; 435/252.3; 435/320.1; 536/23.2

(57) ABSTRACT

The invention relates to an isolated polynucleotide containing a polynucleotide sequence selected from the group

a) polynucleotide that is at least 70% identical to a polynucleotide coding for a polypeptide that contains the amino acid sequence of SEQ ID No. 2,

b) polynucleotide that codes for a polypeptide containing an amino acid sequence that is at least 70% identical to the amino acid sequence of SEQ ID No. 2,

c) polynucleotide that is complementary to the polynucleotides of a) or b), and

d) polynucleotide containing at least 15 successive nucleotides of the polynucleotide sequence of a), b) or c),

and a process for the enzymatic production of L-amino acids using coryneform bacteria in which at least the pepC gene is present in attenuated form, and the use of polynucleotides containing the sequences according to the invention as hybridisation probes.

The subject of the invention are nucleotide sequences from coryneform bacteria coding for the pepC gene and a process for the enzymatic production of amino acids using bacteria in which the pepC gene is attenuated.

### PRIOR ART

L-amino acids, in particular L-lysine, are used in human medicine and in the pharmaceutical industry, in the foodstuffs industry, and most particularly in animal nutrition.

It is known that amino acids can be produced by fermentation of strains of coryneform bacteria, in particular Corynebacterium glutamicum. On account of the great importance of these amino acids constant efforts are being made to improve the production processes. Improvements in production processes may involve fermentation technology measures, such as for example stirring and provision of oxygen, or the composition of the nutrient media, such as for example the sugar concentration during the fermentation, or the working up to the product form by for example ion exchange chromatography, or the intrinsic performance properties of the microorganism itself.

Methods involving mutagenesis, selection and mutant selection are used to improve the performance properties of these microorganisms. In this way strains are obtained that are resistant to antimetabolites or are auxotrophic for regulatorily significant metabolites and that produce amino acids.

For some years recombinant DNA technology methods have also been used to improve strains of Corynebacterium producing L-amino acids, by amplifying individual amino acid biosynthesis genes and investigating the effect on amino acid production.

### OBJECT OF THE INVENTION

The inventors have set themselves the task of developing new procedures for the improved enzymatic production of amino acids.

### DESCRIPTION OF THE INVENTION

The terms L-amino acids or amino acids used hereinafter are understood to mean one or more amino acids, including their salts, selected from the group comprising L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine. L-lysine is particularly preferred.

When L-lysine or lysine is mentioned hereinafter, this accordingly covers not only the bases, but also the salts such as for example lysine monohydrochloride or lysine sulfate.

The present invention provides an isolated polynucleotide from coryneform bacteria containing a polynucleotide sequence coding for the pepC gene, selected from the group

a) polynucleotide that is at least 70% identical to a polynucleotide coding for a polypeptide that contains the amino acid sequence of SEQ ID No. 2,

b) polynucleotide coding for a polypeptide that contains an amino acid sequence that is at least 70% identical to the amino acid sequence of SEQ ID No. 2,

c) polynucleotide that is complementary to the polynucleotides of a) or b), and

d) polynucleotide containing at least 15 successive nucleotides of the polynucleotide sequence of a), b) or c),

the polypeptide preferably having the activity of aminopeptidase I.

The present invention also provides the aforementioned polynucleotide, which is preferably a replicable DNA containing:

(i) the nucleotide sequence shown in SEQ ID No.1, or

(ii) at least one sequence that corresponds to the sequence (i) within the region of degeneration of the genetic code, or

(iii) at least one sequence that hybridises with the sequences that are complementary to the sequences (i) or (ii), and optionally

(iv) functionally neutral sense mutations in (i).

The invention furthermore provides:

a replicable polynucleotide, in particular DNA, containing the nucleotide sequence as illustrated in SEQ ID No.1;

a polynucleotide coding for a polypeptide that contains the amino acid sequence as is illustrated in SEQ ID No. 2;

a vector containing parts of the polynucleotide according to the invention, but at least 15 successive nucleotides of the claimed sequence,

and coryneform bacteria in which the pepC gene is attenuated, in particular by an insertion or deletion.

The present invention likewise provides polynucleotides that consist substantially of a polynucleotide sequence that can be obtained by screening by hybridising a corresponding gene library of a coryneform bacterium that contains the complete gene or parts thereof, using a probe that contains the sequence of the polynucleotide according to the invention according to SEQ ID No.1 or a fragment thereof, and isolating the aforementioned polynucleotide sequence.

Polynucleotides that contain the sequences according to the invention are suitable as hybridisation probes for RNA, cDNA and DNA in order to isolate nucleic acids, polynucleotides or genes in their full length that code for aminopeptidase I, or to isolate those nucleic acids, polynucleotides or genes that exhibit a high degree of similarity to the sequence of the pepC gene.

Polynucleotides that contain the sequences according to the invention are moreover suitable as primers with the aid of which and by employing the polymerase chain reaction (PCR), DNA of genes can be obtained that code for the aminopeptidase I.

Such oligonucleotides serving as probes or primers contain at least 30, preferably at least 20, and most particularly preferably at least 15 successive nucleotides. Also suitable are oligonucleotides having a length of at least 40 or 50 nucleotides.

“Isolated” denotes separated from its natural environment.

“Polynucleotide” refers in general to polyribonucleotides and polydeoxyribonucleotides, which may either be unmodified RNA or DNA or modified RNA or DNA.

The polynucleotides according to the invention include a polynucleotide sequence according to SEQ ID No. 1 or a fragment produced therefrom as well as those that are at least 70%, preferably at least 80% and particularly preferably at least 90% to 95% identical to the polynucleotide according to SEQ ID No. 1 or a fragment produced therefrom.

The term “polypeptides” is understood to mean peptides or proteins that contain two or more amino acids bound via peptide bonds.

The polypeptides according to the invention include a polypeptide according to SEQ ID No. 2, in particular those having the biological activity of aminopeptidase I and also those that are at least 70%, preferably at least 80% and particularly preferably at least 90% to 95% identical to the polypeptide according to SEQ ID No. 2 and that exhibit the aforementioned activity.

The invention furthermore relates to a process for the enzymatic production of amino acids selected from the group comprising L-asparagine, L-threonine, L-serine, L-glutamate, L-glycine, L-alanine, L-cysteine, L-valine, L-methionine, L-isoleucine, L-leucine, L-tyrosine, L-phenylalanine, L-histidine, L-lysine, L-tryptophan and L-arginine, using coryneform bacteria that in particular already produce amino acids and in which the nucleotide sequences coding for the pepC gene are attenuated, in particular switched off or expressed at a low level.

The term “attenuation” used in this context denotes the reduction or switching off of the intracellular activity of one or more enzymes (proteins) in a microorganism that are coded by the corresponding DNA, by for example using a weak promoter or using a gene or allele that codes for a corresponding gene having a low activity or that inactivates the corresponding gene or enzyme (protein), and optionally combining these measures.

The microorganisms that are the subject of the present invention may produce amino acids from glucose, sucrose, lactose, fructose, maltose, molasses, starch, cellulose or from glycerol and ethanol. These microorganisms may be representatives of coryneform bacteria, in particular of the genus Corynebacterium. In the genus Corynebacterium the species Corynebacterium glutamicum should in particular be mentioned, which is known to those skilled in the art for its ability to produce L-amino acids.

Suitable strains of the genus Corynebacterium, in particular of the species Corynebacterium glutamicum (C. glutamicum), are in particular the known wild type strains

Corynebacterium glutamicum ATCC13032

Corynebacterium acetoglutamicum ATCC15806

Corynebacterium acetoacidophilum ATCC13870

Corynebacterium melassecola ATCC17965

Corynebacterium thermoaminogenes FERM BP-1539

Brevibacterium flavum ATCC14067

Brevibacterium lactofermentum ATCC13869 and

Brevibacterium divaricatum ATCC14020

and mutants or strains derived therefrom that produce L-amino acids.

The new pepC gene coding for aminopeptidase I has been isolated from C. glutamicum.

In order to isolate the pepC gene or also other genes from C. glutamicum, a gene library of this microorganism is first of all introduced into Escherichia coli (E. coli). The introduction of gene libraries is described in generally known textbooks and manuals. As an example there may be mentioned the textbook by Winnacker: Gene and Klone, Eine Einführung in die Gentechnologie (Verlag Chemie, Weinheim, Germany, 1990), or the manual by Sambrook et al.: Molecular Cloning, A Laboratory Manual (Cold Spring Harbor Laboratory Press, 1989). A very well-known gene library is that of the E. coli K-12 strain W3110, which was introduced by Kohara et al. (Cell 50, 495-508 (1987)) into λ-vectors. Bathe et al. (Molecular and General Genetics, 252:255-265, 1996) describe a gene library of C. glutamicum ATCC13032, which was introduced by means of the cosmid vector SuperCos I (Wahl et al., 1987, Proceedings of the National Academy of Sciences USA, 84:2160-2164) in the E. coli K-12 strain NM554 (Raleigh et al., 1988, Nucleic Acids Research 16:1563-1575).

Börmann et al. (Molecular Microbiology 6(3), 317-326 (1992)) again describe a gene library of C. glutamicum ATCC13032 using the cosmid pHC79 (Hohn and Collins, 1980, Gene 11, 291-298).

In order to produce a gene library of C. glutamicum in E. coli there may also be used plasmids such as pBR322 (Bolivar, 1979, Life Sciences, 25, 807-818) or pUC9 (Vieira et al., 1982, Gene, 19:259-268). Suitable hosts are in particular those E. coli strains that are restriction and recombinant defective, such as for example the strain DH5αmcr, which has been described by Grant et al. (Proceedings of the National Academy of Sciences USA, 87 (1990) 4645-4649). The long DNA fragments cloned with the aid of cosmids or other λ-vectors may subsequently be sub-cloned into customary vectors suitable for DNA sequencing and then sequenced, such as is described for example by Sanger et al. (Proceedings of the National Academy of Sciences of the United States of America, 74:5463-5467, 1977).

The DNA sequences obtained may then be investigated with known algorithms or sequence analysis programs, such as for example that of Staden (Nucleic Acids Research 14, 217-232(1986)), that of Marck (Nucleic Acids Research 16, 1829-1836 (1988)) or the GCG program of Butler (Methods of Biochemical Analysis 39, 74-97 (1998)).

The new DNA sequence of C. glutamicum coding for the pepC gene has been found, and as SEQ ID No. 1 is covered by the present invention. The amino acid sequence of the corresponding protein has furthermore been derived from the existing DNA sequence using the aforedescribed methods. The resultant amino acid sequence of the pepC gene product is represented in SEQ ID No. 2.

Coding DNA sequences that are obtained from SEQ ID No. 1 as a result of the degenerability of the genetic code are also covered by the invention. Similarly, DNA sequences that hybridise with SEQ ID No. 1 or parts of SEQ ID No. 1 are also covered by the invention. Furthermore, in this specialist field conservative amino acid replacements, such as for example the replacement of glycine by alanine or of aspartic acid by glutamic acid in proteins, are known as sense mutations, which do not lead to any fundamental change in the activity of the protein, i.e. are functionally neutral. Furthermore, it is known that changes at the N-terminus and/or C-terminus of a protein do not significantly impair or may even stabilise its function. Those skilled in the art can find details of this in, inter alia, Ben-Bassat et al. (Journal of Bacteriology 169:751-757 (1987)), in O'Regan et al. (Gene 77:237-251 (1989)), in Sahin-Toth et al. (Protein Sciences 3:240-247 (1994)), in Hochuli et al. (Bio/Technology 6:1321-1325 (1988)) and in known textbooks of genetics and molecular biology. Amino acid sequences that are obtained in a corresponding manner from SEQ ID No. 2 are likewise covered by the invention.

Similarly, DNA sequences that hybridise with SEQ ID No. 1 or parts of SEQ ID No. 1 are covered by the invention. Finally, DNA sequences that are produced from SEQ ID No. 1 by means of the polymerase chain reaction (PCR) using primers are covered by the invention. Such oligonucleotides typically have a length of at least 15 nucleotides.

The person skilled in the art can find details of the identification of DNA sequences by means of hybridisation in, inter alia, the textbook “The DIG System User's Guide for Filter Hybridization” published by Boehringer Mannheim GmbH (Mannheim, Germany, 1993) and in Liebl et al. (International Journal of Systematic Bacteriology 41: 255-260 (1991)). The hybridisation takes place under stringent conditions, i.e. only hybrids are formed in which the probe and target sequence, i.e. the polynucleotides treated with the probe, are at least 70% identical. It is known that the stringency of the hybridisation, including the wash stage, is influenced or determined by varying the buffer composition, the temperature and the salt concentration. The hybridisation reaction is preferably carried out under conditions of relatively low stringency compared to the wash stages (Hybaid Hybridisation Guide, Hybaid Limited, Teddington, UK, 1996).

A 5× SSC-buffer may for example be used at a temperature of ca. 50° C.-68° C. for the hybridisation reaction. In this connection probes may also hybridise with polynucleotides that are less than 70% identical to the sequence of the probe. Such hybrids are less stable and are removed by washing under stringent conditions. This may be achieved for example by lowering the salt concentration to 2× SSC and optionally subsequently to 0.5× SSC (The DIG System User's Guide for Filter Hybridisation, Boehringer Mannheim, Mannheim, Germany, 1995), the temperature being adjusted to ca. 50° C.-68° C. It is also possible to reduce the salt concentration down to 0.1× SSC. By stepwise raising of the hybridisation temperature in steps of ca. 1-2° C. from 50° C. to 68° C., polynucleotide fragments can be isolated that are for example at least 70% or at least 80% or at least 90% to 95% identical to the sequence of the probe that is employed. Further details concerning the hybridisation are available on the market in the form of so-called kits (e.g. DIG Easy Hyb from Roche Diagnostics GmbH, Mannheim, Germany, Catalogue No. 1603558).

The person skilled in the art can find details of the amplification of DNA sequences by means of the polymerase chain reaction (PCR) in, inter alia, the manual by Gait: Oligonucleotides: A Practical Approach (IRL Press, Oxford, UK, 1984) and in Newton and Graham: PCR (Spektrum Akademischer Verlag, Heidelberg, Germany, 1994).

It has been found that coryneform bacteria after attenuation of the pepC gene produce amino acids in an improved manner.

In order to achieve an attenuation, either the expression of the pepC gene or the catalytic properties of the enzyme protein may be reduced or switched off. Optionally both measures may be combined.

The reduction of the gene expression may be achieved by suitable culture conditions or by genetic alteration (mutation) of the signal structures of the gene expression. Signal structures of the gene expression are for example repressor genes, activator genes, operators, promoters, attenuators, ribosome binding sites, the start codon and terminators. The person skilled in the art can obtain further information on this in for example patent application WO 96/15246, in Boyd and Murphy (Journal of Bacteriology 170: 5949 (1988)), in Voskuil and Chambliss (Nucleic Acids Research 26: 3548 (1998), in Jensen and Hammer (Biotechnology and Bioengineering 58: 191 (1998)), in Pátek et al. (Microbiology 142: 1297 (1996)), Vasicova et al. (Journal of Bacteriology 181: 6188 (1999)) and in known textbooks of genetics and molecular biology, such as for example the textbook by Knippers (“Molekulare Genetik”, 6th Edition, Georg Thieme Verlag, Stuttgart, Germany, 1995) or the textbook by Winnacker (“Gene and Klone”, VCH Verlagsgesellschaft, Weinheim, Germany, 1990).

Mutations that lead to an alteration or reduction of the catalytic properties of enzyme proteins are known in the prior art; as examples there may be mentioned the work of Qiu and Goodman (Journal of Biological Chemistry 272: 8611-8617 (1997)), Sugimoto et al. (Bioscience Biotechnology and Biochemistry 61: 1760-1762 (1997)) and Möckel (“Die Threonindehydratase aus Corynebacterium glutamicum: Aufhebung der allosterischen Regulation and Struktur des Enzyms”, and reports published by the Jülich Research Centre, Jül-2906, ISSN09442952, Jülich, Germany, 1994). Overviews may be obtained from known textbooks on genetics and molecular biology, for example that of Hagemann (“Allgemeine Genetik”, Gustav Fischer Verlag, Stuttgart, 1986).

Mutations in the present context include transitions, transversions, insertions and deletions. Depending on the effect of the amino acid replacement on the enzyme activity, one talks either of missense mutations or nonsense mutations. Insertions or deletions of at least one base pair (bp) in a gene lead to frame shift mutations, following which false amino acids are incorporated or the translation terminates prematurely. Deletions of several codons typically lead to a complete cessation of enzyme activity. Details of the production of such mutations are part of the prior art and may be obtained from known textbooks on genetics and molecular biology, such as for example the textbook by Knippers (“Molekulare Genetik”, 6^{th} Edition, Georg Thieme Verlag, Stuttgart, Germany, 1995), the textbook by Winnacker (“Gene and Klone”, VCH Verlagsgesellschaft, Weinheim, Germany, 1990) or the textbook by Hagemann (“Allgemeine Genetik”, Gustav Fischer Verlag, Stuttgart, 1986).

A conventional method of mutating genes of C. glutamicum is the method of gene disruption and gene replacement described by Schwarzer and Pühler (Bio/Technology 9, 84-87 (1991)).

In the method of gene disruption a central part of the coding region of the gene in question is cloned into a plasmid vector that can replicate in a host (typically E. coli), but not in C. glutamicum. Suitable vectors are for example pSUP301 (Simon et al., Bio/Technology 1, 784-791 (1983)), pK18mob or pK19mob (Schäfer et al., Gene 145, 69-73 (1994)), pK18mobsacB or pK19mobsacB (Jäger et al., Journal of Bacteriology 174: 5462-65 (1992)), pGEM-T (Promega Corporation, Madison, Wis., USA), pCR2.1-TOPO (Shuman (1994), Journal of Biological Chemistry 269:32678-84; U.S. Pat. No. 5,487,993), pCR®Blunt (Invitrogen, Groningen, Netherlands; Bernard et al., Journal of Molecular Biology, 234: 534-541 (1993)) or pEM1 (Schrumpf et al, 1991, Journal of Bacteriology 173:4510-4516). The plasmid vector that contains the central part of the coding region of the gene is then converted by conjugation or transformation into the desired strain of C. glutamicum. The method of conjugation is described for example by Schäfer et al. (Applied and Environmental Microbiology 60, 756-759 (1994)). Methods of transformation are described for example in Thierbach et al. (Applied Microbiology and Biotechnology 29, 356-362 (1988)), Dunican and Shivnan (Bio/Technology 7, 1067-1070 (1989)) and Tauch et al. (FEMS Microbiological Letters 123, 343-347 (1994)). After homologous recombination by means of a cross-over event, the coding region of the relevant gene is disrupted by the vector sequence and two incomplete alleles are obtained, missing respectively the 3′- and 5′-end. This method has been used for example by Fitzpatrick et al. (Applied Microbiology and Biotechnology 42, 575-580 (1994)) to switch off the recA gene of C. glutamicum.

In the gene replacement method a mutation, such as for example a deletion, insertion or base replacement, is produced in vitro in the gene that is of interest. The resultant allele is in turn cloned into a non-replicative vector for C. glutamicum, and this is then converted by transformation or conjugation into the desired host of C. glutamicum. After homologous recombination by means of a first cross-over event effecting integration, and an appropriate second cross-over event effecting an excision, the incorporation of the mutation or allele in the target gene or in the target sequence is achieved. This method has been used for example by Peters-Wendisch et al. (Microbiology 144, 915-927 (1998)) to switch off the pyc gene of C. glutamicum by a deletion.

A deletion, insertion or a base replacement can be incorporated intp the pepC gene in this way.

In addition, for the production of L-amino acid it may be advantageous, besides attenuating the pepC gene, to enhance, in particular overexpress, one or more enzymes of the relevant biosynthesis pathway, glycolysis, anaplerosis, citric acid cycle, pentose phosphate cycle, amino acid export and optionally regulatory proteins.

The term “enhancement” describes in this connection the raising of the intracellular activity of one or more enzymes (proteins) in a microorganism that are coded by the corresponding DNA, by for example increasing the number of copies of the gene or genes, using a strong promoter, or using a gene or allele that codes for the corresponding enzyme (protein) with a high activity, and optionally by a combination of these measure.

Thus for example, for the preparation of L-amino acids, besides attenuating the pepC gene, one or more of the genes selected from the following group is simultaneously enhanced, in particular overexpressed:

the gene dapA coding for dihydrodipicolinate synthase (EP-B 0 197 335)

the gene gap coding for glyceraldehyde-3-phosphate dehydrogenase (Eikmanns (1992), Journal of Bacteriology 174: 6076-6086),

the gene tpi coding for triose phosphate isomerase (Eikmanns (1992), Journal of Bacteriology 174:6076-6086),

the gene pgk coding for 3-phosphoglycerate kinase (Eikmanns (1992), Journal of Bacteriology 174:6076-6086),

the gene zwf coding for glucose-6-phosphate dehydrogenase (JP-A-09224661),

the gene pyc coding for pyruvate-carboxylase (DE-A-198 31 609),

the gene mqo coding for malate-quinone-oxidoreductase (Molenaar et al., European Journal of Biochemistry 254, 395-403 (1998)),

the gene lysC coding for a feedback-resistant aspartate kinase (EP-B-0387527; EP-A-0699759; WO 00/63388),

the gene lysE coding for lysine export (DE-A-195 48 222),

the gene hom coding for homoserine dehydrogenase (EP-A 0131171),

the gene ilvA coding for threonine dehydratase (Möckel et al., Journal of Bacteriology (1992) 8065-8072)) or the allele ilvA(Fbr) coding for a “feedback-resistant” threonine dehydratase (Möckel et al., (1994) Molecular Microbiology 13: 833-842),

the gene ilvBN coding for acetohydroxy acid synthase (EP-B 0356739),

the gene ilvD coding for dihydroxy acid dehydratase (Sahm and Eggeling (1999) Applied and Environmental Microbiology 65: 1973-1979),

the gene zwal coding for the zwal protein (DE: 19959328.0, DSM 13115).

Moreover it may be advantageous for the production of amino acids, besides attenuating the pepC gene, at the same time to attenuate, in particular to reduce the expression, of one or more of the genes selected from the group:

the gene pck coding for phosphoenol pyruvate carboxykinase (DE 199 50 409.1, DSM 13047),

the gene pgi coding for glucose-6-phosphate isomerase (U.S. Ser. No. 09/396,478, DSM 12969),

the gene poxB coding for pyruvate oxidase (DE:1995 1975.7, DSM 13114),

the gene zwa2 coding for the zwa2 protein (DE: 19959327.2, DSM 13113).

Moreover it may be advantageous for the production of amino acids, besides attenuating the pepC gene, to switch off undesired secondary reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms produced according to the invention are likewise covered by the invention and for the purposes of producing L-amino acids may be cultivated continuously or batchwise in a batch process, or in a feed batch process or repeated batch process. A summary of known cultivation methods is described in the textbook by Chmiel (Bioprozesstechnik 1. Einführung in die Bioverfahrens-technik (Gustav Fischer Verlag, Stuttgart, 1991)) or in the textbook by Storhas (Bioreaktoren and periphere Einrichtungen (Vieweg Verlag, Brunswick/Wiesbaden, 1994)).

The culture medium to be used must satisfy in an appropriate manner the requirements of the respective strains. Descriptions of culture media for various microorganisms are given in the handbook “Manual of Methods for General Bacteriology” of the American Society for Bacteriology (Washington D.C., USA, 1981).

As carbon source there may be used sugars and carbohydrates such as for example glucose, sucrose, lactose, fructose, maltose, molasses, starch and cellulose, oils and fats such as for example soya oil, sunflower oil, groundnut oil and coconut oil, fatty acids such as for example palmitic acid, stearic acid and linoleic acid, alcohols such as for example glycerol and ethanol, and organic acids such as for example acetic acid. These substances may be used individually or as a mixture.

As nitrogen source there may be used organic nitrogen-containing compounds such as peptones, yeast extract, meat extract, malt extract, corn steep liquor, soya bean flour and urea, or inorganic compounds such as ammonium sulfate, ammonium chloride, ammonium phosphate, ammonium carbonate and ammonium nitrate. The nitrogen sources may be used individually or as a mixture.

As phosphorus source there may be used phosphoric acid, potassium dihydrogen phosphate or dipotassium hydrogen phosphate, or the corresponding sodium-containing salts. The culture medium must furthermore contain salts of metals, such as for example magnesium sulfate or iron sulfate, that are necessary for growth. Finally, essential growth promoters such as amino acids and vitamins may in addition be added to the aforementioned substances. Suitable precursors may moreover be added to the culture medium. The aforementioned starting substances may be added to the culture in the form of a single batch, or metered in in an appropriate manner during the cultivation procedure.

In order to control the pH of the culture basic compounds such as sodium hydroxide, potassium hydroxide, ammonia or ammonia water, or acidic compounds such as phosphoric acid or sulfuric acid may be added in an appropriate manner. In order to control foam formation anti-foaming agents such as for example fatty acid polyglycol esters may be used. In order to maintain the stability of plasmids selectively acting substances, such as for example antibiotics, may be added to the medium. In order to maintain aerobic conditions, oxygen or oxygen-containing gas mixtures, such as for example air, are pumped into the culture. The temperature of the culture is normally 20° C. to 45° C. and preferably 25° C. to 40° C. The cultivation is continued until a maximum amount of the desired product has been formed. This target is normally reached within 10 hours to 160 hours.

Methods for determining L-amino acids are known from the prior art. The analysis may for example be carried out as described by Spackman et al. (Analytical Chemistry, 30, (1958), 1190) by anion exchange chromatography followed by ninhydrin derivatisation, or it may be carried out by reversed phase HPLC, as described by Lindroth et al. (Analytical Chemistry (1979) 51: 1167-1174).

The process according to the invention serves for the enzymatic production of amino acids.

The following microorganism was filed as a pure culture according to the Budapest Convention on 17.01.2001 at the German Collection for Microorganisms and Cell Cultures (DSMZ, Brunswick, Germany):

Escherichia coli Top10/pCR2.1pepCint as DSM 13985.

The present invention is illustrated in more detail hereinafter with the aid of examples of implementation.

The isolation of plasmid DNA from Escherichia coli as well as all techniques for the restriction, Klenow and alkaline phosphatase treatment were carried out according to Sambrook et al. (Molecular Cloning. A Laboratory Manual, 1989, Cold Spring Harbour Laboratory Press, Cold Spring Harbor, N.Y., USA). Methods for the transformation of Escherichia coli are likewise described in this handbook.

The compositions of conventional nutrient media such as LB medium or TY medium may also be obtained from the handbook by Sambrook et al.

### EXAMPLE 1

Production of a Genomic Cosmid Gene Library from C. glutamicum ATCC 13032

Chromosomal DNA from C. glutamicum ATCC 13032 was isolated as described by Tauch et al., (1995, Plasmid 33:168-179) and partially cleaved with the restriction enzyme Sau3AI (Amersham Pharmacia, Freiburg, Germany, Product Description Sau3AI, Code no. 27-0913-02). The DNA fragments were dephosphorylated with shrimp alkaline phosphatase (Roche Molecular Biochemicals, Mannheim, Germany, Product Description SAP, Code no. 1758250). The DNA of the cosmid vector SuperCos1 (Wahl et al. (1987), Proceedings of the National Academy of Sciences, USA 84:2160-2164), obtained from Stratagene (La Jolla, USA, Product Description SuperCos1 Cosmid Vektor Kit, Code no. 251301) was cleaved with the restriction enzyme XbaI (Amersham Pharmacia, Freiburg, Germany, Product Description XbaI, Code no. 27-0948-02) and likewise dephosphorylated with shrimp alkaline phosphatase.

The cosmid DNA was then cleaved with the restriction enzyme BamHI (Amersham Pharmacia, Freiburg, Germany, Product Description BamHI, Code no. 27-0868-04). The cosmid DNA treated in this way was mixed with the treated ATCC13032-DNA, and the batch was then treated with T4-DNA ligase (Amersham Pharmacia, Freiburg, Germany, Product Description T4-DNA ligase, Code no.27-0870-04). The ligation mixture was then packed into phages using Gigapack II XL Packing Extracts (Stratagene, La Jolla, USA, Product Description Gigapack II XL Packing Extract, Code no. 200217).

In order to infect the E. coli strain NM554 (Raleigh et al. 1988, Nucleic Acid Res. 16:1563-1575) the cells were taken up in 10 mM MgSO₄ and mixed with an aliquot of the phage suspension. Infection and titration of the cosmid library were carried out as described by Sambrook et al. (1989, Molecular Cloning: A Laboratory Manual, Cold Spring Harbor), the cells being plated out on LB agar (Lennox, 1955, Virology, 1:190)+100 μg/ml ampicillin. After incubation overnight at 37° C. recombinant individual clones were selected.

### EXAMPLE 2

Isolation and Sequencing of the Gene pepC

The cosmid DNA of an individual colony was isolated with the Qiaprep Spin Miniprep Kit (Product No. 27106, Qiagen, Hilden, Germany) according to the manufacturer's instructions and then partially cleaved with the restriction enzyme Sau3AI (Amersham Pharmacia, Freiburg, Germany, Product Description Sau3AI, Product No. 27-0913-02). The DNA fragments were dephosphorylated with shrimp alkaline phosphatase (Roche Molecular Biochemicals, Mannheim, Germany, Product Description SAP, Product No. 1758250). After gel electrophoresis separation the cosmid fragments were isolated in the size range from 1500 to 2000 bp using the QiaExII Gel Extraction Kit (Product No. 20021, Qiagen, Hilden, Germany).

The DNA of the sequencing vector pZero-1 obtained from Invitrogen (Groningen, Netherlands, Product Description Zero Background Cloning Kit, Product No. K2500-01) was cleaved with the restriction enzyme BamHI (Amersham Pharmacia, Freiburg, Germany, Product Description BamHI, Product No. 27-0868-04). The ligation of the cosmid fragments into the sequencing vector pZero-l was carried out as described by Sambrook et al. (1989, Molecular Cloning: A Laboratory Manual, Cold Spring Harbor), the DNA mixture having been incubated overnight with T4 ligase (Pharmacia Biotech, Freiburg, Germany). This ligation mixture was then electoporated into the E. coli strain DH5αMCR (Grant, 1990, Proceedings of the National Academy of Sciences, U.S.A., 87:4645-4649) (Tauch et al. 1994, FEMS Microbiol. Letters, 123:343-7) and plated out onto LB-agar (Lennox, 1955, Virology, 1:190) with 50 mg/l zeocin ausplattiert.

The plasmid preparation of the recombinant clone was carried out with the Biorobot 9600 (Product No. 900200, Qiagen, Hilden, Germany). The sequencing was performed according to the dideoxy chain termination method of Sanger et al. (1977, Proceedings of the National Academies of Sciences, U.S.A., 74:5463-5467) as modified by Zimmermann et al. (1990, Nucleic Acids Research, 18:1067). The “RR dRhodamin Terminator Cycle Sequencing Kit” from PE Applied Biosystems (Product No. 403044, Weiterstadt, Germany) was used. The gel electrophoresis separation and analysis of the sequencing reaction were carried out in a “Rotiphoresis NF Acrylamide/Bisacrylamide” Gel (29:1) (Product No. A124.1, Roth, Karlsruhe, Germany) using the “ABI Prism 377” sequencing device from PE Applied Biosystems (Weiterstadt, Germany).

The crude sequence data thus obtained were then processed using the Staden Program Packet (1986, Nucleic Acids Research, 14:217-231) Version 97-0. The individual sequences of the pZero1 derivates were assembled to form a coherent contig. The computer-assisted coding region analysis was prepared using the XNIP program (Staden, 1986, Nucleic Acids Research, 14:217-231). Further analyses were carried out with the “BLAST search programs” (Altschul et al., 1997, Nucleic Acids Research, 25:33893402) against the non-redundant database of the National Center for Biotechnology Information (NCBI, Bethesda, Md., USA).

The nucleotide sequence thus obtained is represented in SEQ ID No. 1. Analysis of the nucleotide sequence revealed an open reading frame of 1263 bp, which was designated pepC gene. The pepC gene codes for a polypeptide of 420 amino acids.

### EXAMPLE 3

Production of an Integration Vector for the Integration Mutagenesis of the pepC Gene

Chromosomal DNA was isolated from the strain ATCC 13032 by the method of Eikmanns et al. (Microbiology 140: 1817-1828 (1994)). On account of the sequence of the pepC gene known from Example 2 for C. glutamicum, the following oligonucleotides were selected for the polymerase chain reaction (see SEQ ID No. 3 and SEQ ID No. 4):

pepC-int1:
5′ CTT TCC TCA CAC GGT TGG 3′
pepC-int2:
5′ TCC CAC TTC TTC ATG ATC G 3′

The represented primers were synthesised by MWG Biotech (Ebersberg, Germany) and the PCR reaction was carried out according to the standard PCR method of Innis et al. (PCR protocols. A guide to methods and applications, 1990, Academic Press) using Taq polymerase from Boehringer Mannheim (Germany, Product Description Taq DNA Polymerase, Product No. 1 146 165). With the aid of the polymerase chain reaction the primers permit the amplification of a 504 bp large internal fragment of the pepC gene. The thus amplified product was tested electrophoretically in a 0.8% agarose gel.

The amplified DNA fragment was ligated into the vector pCR2.1-TOPO (Mead at al. (1991) Bio/Technology 9:657-663) using the TOPO TA Cloning Kit from Invitrogen Corporation (Carlsbad, Calif., USA; Cat. No. K4500-01).

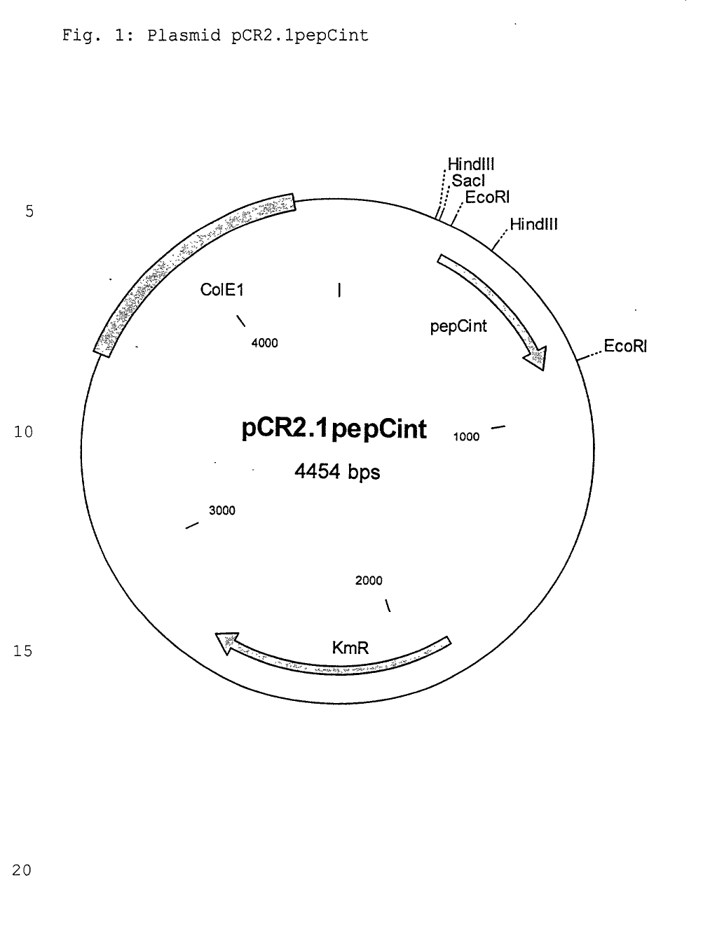

The E. coli strain TOP10 was then electroporated with the ligation batch (Hanahan, In: DNA cloning. A practical approach. Vol. I. IRL-Press, Oxford, Washington DC, USA, 1985). Plasmid-carrying cells were selected by plating out the transformation batch onto LB agar (Sambrook et al., Molecular cloning: a laboratory manual. 2^{nd} Ed. Cold Spring Harbor Laboratory Press, Cold Spring Harbor, N.Y., 1989) that had been supplemented with 50 mg/l of kanamycin. Plasmid DNA was isolated from a transformant using the QIAprep Spin Miniprep Kit from Qiagen and was checked by restriction with the restriction enzyme EcoRI followed by agarose gel electrophoresis (0.8%). The plasmid was named pCR2.1pepCint and is represented in FIG. 1.

The following microorganism was filed according to the Budapest Convention as a pure culture on 17.01.2001 at the German Collection for Microorganisms and Cell Cultures (DSMZ, Brunswick, Germany):

Escherichia coli Top10/pCR2.1pepCint as DSM 13985.

### EXAMPLE 4

Integration Mutagenesis of the pepC Genes in the Strain DSM 5715

The vector pCR2.1pepCint mentioned in Example 3 was electroporated into Corynebacterium glutamicum DSM 5715 according to the electroporation method of Tauch et. al. (FEMS Microbiological Letters, 123:343-347 (1994)). The strain DSM 5715 is an AEC-resistant lysine producer. The vector pCR2.1pepCint cannot replicate independently in DSM5715 and thus only remains in the cell if it has integrated into the chromosome of DSM 5715. The selection of clones with pCR2.1pepCint integrated into the chromosome was made by plating out the electroporation batch onto LB agar (Sambrook et al., Molecular cloning: a laboratory manual. 2^{nd} Ed. Cold Spring Harbor Laboratory Press, Cold Spring Harbor, N.Y.) that had been supplemented with 15 mg/l of kanamycin.

In order to demonstrate the integration the pepCint fragment was labelled using the Dig Hybridisation Kit from Boehringer according to the method described in “The DIG System User's Guide for Filter Hybridization” published by Boehringer Mannheim GmbH (Mannheim, Germany, 1993). Chromosomal DNA of a potential integrant was isolated according to the method of Eikmanns et al. (Microbiology 140: 1817-1828 (1994)) and was in each case cleaved with the restriction enzymes SacI, EcoRI and HindIII. The resultant fragments were separated by means of agarose gel electrophoresis and hybridised at 68° C. using the Dig Hybridisation Kit from Boehringer. The plasmid pCR2.1pepCint mentioned in Example 3 had inserted itself into the chromosome of DSM5715 within the chromosomal pepC gene. The strain was designated DSM5715::pCR2.1pepCint.

### EXAMPLE 5

Production of Lysine

The C. glutamicum strain DSM5715::pCR2.1pepCint obtained in Example 4 was cultivated in a nutrient medium suitable for the production of lysine and the lysine content in the culture supernatant was determined.

For this purpose the strain was first of all incubated for 24 hours at 33° C. on an agar plate with the corresponding antibiotic (brain-heart agar with kanamycin (25 mg/l). Starting from this agar plate culture a preculture was inoculated (10 ml of medium in a 100 ml Erlenmeyer flask). The full medium CgIII was used as medium for the preculture.

| | Medium Cg III | |
|---|---|---|
| NaCl | 2.5 | g/l |
| Bacto-Peptone | 10 | g/l |
| Bacto-Yeast Extract | 10 | g/l |
| Glucose (autoclaved separately) | 2% | (w/v) |
| The pH value was adjusted to pH | | |
| 7.4 | | |

Kanamycin (25 mg/l) was added to this preculture. The preculture was then incubated for 16 hours at 33° C. at 240 rpm on a shaker table. From this preculture a main culture was inoculated so that the initial OD (660 nm) of the main culture was 0.1 OD. The medium MM was used for the main culture.

| | Medium MM | |
|---|---|---|
| CSL (Corn Steep Liquor) | 5 | g/l |
| MOPS | 20 | g/l |
| Glucose (autoclaved separately) | 50 | g/l |
| Salts: | | |
| (NH₄)₂SO₄) | 25 | g/l |
| KH₂PO₄ | 0.1 | g/l |
| MgSO₄.7H₂O | 1.0 | g/l |
| CaCl₂.2H₂O | 10 | mg/l |
| FeSO₄.7H₂O | 10 | mg/l |
| MnSO₄.H₂O | 5.0 | mg/l |
| Biotin (sterile filtered) | 0.3 | mg/l |
| Thiamine.HCl (sterile filtered) | 0.2 | mg/l |
| Leucine (sterile filtered) | 0.1 | g/l |
| CaCO₃ | 25 | g/l |

CSL, MOPS and the salt solution are adjusted with ammonia water to pH 7 and autoclaved. The sterile substrate solutions and vitamin solutions as well as the dry autoclaved CaCO₃ are then added.

Cultivation is carried out in a 10 ml volume in a 100 ml Erlenmeyer flask equipped with baffles. Kanamycin was added (25 mg/l). The cultivation was carried out at 33° C. and 80% atmospheric humidity.

After 72 hours the OD was determined at a measurement wavelength of 660 nm with a Biomek 1000 (Beckmann Instruments GmbH, Munich). The amount of lysine formed was determined by ion exchange chromatography and post-column derivatisation with ninhydrin detection using an amino acid analyser from Eppendorf-BioTronik (Hamburg, Germany).

The results of the experiment are shown in Table 1.

TABLE 1

| | | Lysine-HCl |
|---|---|---|
| Strain | OD (660) | g/l |
| DSM5715 | 8.2 | 13.74 |
| DSM5715::pCR2.1pepCint | 9.2 | 14.17 |

The following figure accompanies the decription:

FIG. 1: Map of the plasmid pCR2.1pepCint.

The acronyms and abbreviations used have the following meanings.

| | |
|---|---|
| KmR: | Kanamycin resistance gene |
| EcoRI: | Cleavage site of the restriction enzyme |
| | EcoRI |
| HindIII: | Cleavage site of the restriction enzyme |
| | HindIII |
| SacI: | Cleavage site of the restriction enzyme |
| | SacI |
| pepCint: | internal fragment of the pepC gene |
| ColE1: | Replication origin of the plasmid ColE1 |

1. An isolated polynucleotide from coryneform bacteria containing a polynucleotide sequence coding for the pepC gene, selected from the group comprising
a) polynucleotide that is at least 70% identical to a polynucleotide coding for a polypeptide that contains the amino acid sequence of SEQ ID No. 2,
b) polynucleotide coding for a polypeptide that contains an amino acid sequence that is at least 70% identical to the amino acid sequence of SEQ Id No. 2,
c) polynucleotide that is complementary to the polynucleotides of a) or b), and
d) polynucleotide containing at least 15 successive nucleotides of the polynucleotide sequence of a), b) or c),
the polypeptide preferably having the activity of aminopeptidase I:

2. A polynucleotide as claimed in claim 1, wherein the polynucleotide is a preferably recombinant DNA replicable in coryneform bacteria.

3. A polynucleotide as claimed in claim 1, wherein the polynucleotide is an RNA.

4. A polynucleotide as claimed in claim 2, containing the nucleic acid sequence as represented in SEQ ID No. 1.

5. Replicable DNA as claimed in claim 2, containing
(i) the nucleotide sequence shown in SEQ ID No. 1, or
(ii) at least one sequence that corresponds to the sequence (i) within the region of degeneration of the genetic code, or
(iii) at least one sequence that hybridises with the sequence complementary to the sequence (i) or (ii), and optionally
(iv) functionally neutral sense mutations in (i).

6. Replicable DNA as claimed in claim 2, wherein the hybridisation is carried out under a stringency corresponding to at most 2× SSC.

7. A polynucleotide sequence as claimed in claim 1 that codes for a polypeptide containing the amino acid sequence represented in SEQ ID No. 2.

8. An integration vector pCR2.1pepCint which
8.1 carries a 504 bp large internal fragment of the pepC gene,
8.2 whose restriction site is reproduced in FIG. 1, and
8.3 which in the E. coli strain Top10/pCR2.1pepCint is filed under No. DSM 13985 at the German Collection for Microorganisms and Cell Cultures.

9. Coryneform bacteria in which the pepC gene is attenuated, in particular switched off, preferably by deletion.

10. A process for the enzymatic production of L-amino acid, in particular L-lysine, wherein the following steps are carried out:
a) fermentation of the coryneform bacteria producing the desired L-amino acid, in which at least the pepC gene or nucleotide sequences coding for the latter is/are attenuated, in particular switched off;
b) accumulation of the L-amino acid in the medium or in the cells of the bacteria, and
c) isolation of the L-amino acid.

11. A process as claimed in claim 10, wherein bacteria are used in which in addition further genes of the biosynthesis pathway of the desired L-amino acid are enhanced.

12. A process as claimed in claim 10, wherein bacteria are used in which the metabolic pathways that reduce the formation of the desired L-amino acid are at least partially switched off.

13. A process as claimed in claim 10, wherein the expression of the polynucleotide(s) that codes/code for the pepC gene is attenuated, in particular is switched off.

14. A process as claimed in claim 10, wherein the catalytic properties of the polypeptide (enzyme protein) for which the polynucleotide pepC codes are reduced.

15. A process as claimed in claim 10, wherein for the production of L-amino acids coryneform microorganisms are fermented, in which at the same time one or more of the genes selected from the following group is/are enhanced or overexpressed
15.1 the gene dapA coding for dihydrodipicolinate synthase,
15.2 the gene gap coding for glyceraldehyde-3-phosphate dehydrogenase,
15.3 the gene tpi coding for triosephosphate isomerase,
15.4 the gene pgk coding for 3-phosphoglycerate kinase,
15.5 the gene zwf coding for glucose-6-phosphate dehydrogenase,
15.6 the gene pyc coding for pyruvate carboxylase,
15.7 the gene mqo coding for malate-quinone oxidoreductase,
15.8 the gene lysC coding for a feedback-resistant aspartate kinase,
15.9 the gene lysE coding for lysine export,
15.10 the gene hom coding for homoserine dehydrogenase,
15.11 the gene ilvA coding for threonine dehydratase or the allele ilvA(Fbr) coding for a feedback-resistant threonine dehydratase,
15.12 the gene ilvBN coding for acetohydroxy acid synthase,
15.13 the gene ilvD coding for dihydroxy acid dehydratase,
15.14 the gene zwal coding for the zwal protein.

16. A process as claimed in claim 10, wherein for the production of L-amino acids coryneform microorganisms are fermented in which at the same time one or more of the genes selected from the following group is/are attenuated
16.1 the gene pck coding for phosphoenol pyruvate carboxykinase,
16.2 the gene pgi coding for glucose-6-phosphate isomerase,
16.3 the gene poxB coding for pyruvate oxidase,
16.4 the gene zwa2 coding for the zwa2 protein.

17. Coryneform bacteria containing a vector that carries parts of the polynucleotide according to claim 1, but at least 15 successive nucleotides of the claimed sequence.

18. A process as claimed in one or more of the preceding claims, wherein microorganisms of the species Corynebacterium glutamicum are used.

19. A process for detecting RNA, cDNA and DNA in order to isolate nucleic acids or polynucleotides or genes that code for aminopeptidase I or that have a high degree of similarity to the sequence of the pepC gene, wherein the polynucleotide containing the polynucleotide sequences as claimed in claims 1, 2, 3 or 4 is used as hybridisation probes.
